(19) United States
(12) Patent Application Publication
(10) Pub. No.: US 2022/0313240 A1
(43) Pub. Date: Oct. 6, 2022

(54) GASTROINTESTINAL SUTURE RETENTION SYSTEM

(71) Applicant: Celero Systems, Inc., Lincoln, MA (US)

(72) Inventors: Benjamin David Pless, Lincoln, MA (US); Daniel Bacher, Walpole, MA (US)

(21) Appl. No.: 17/709,516

(22) Filed: Mar. 31, 2022

Related U.S. Application Data
(60) Provisional application No. 63/169,419, filed on Apr. 1, 2021.
(60) Provisional application No. 63/169,439, filed on Apr. 1, 2021.

(51) Int. Cl. A61B 17/04

(52) U.S. Cl. CPC A61B 17/0401; A61B 17/0469; A61B 17/0482; A61B 2017/06052

(57) ABSTRACT

Endoscopic and self-deploying gastrointestinal suture retention systems are provided herein. Such systems provide prolonged residency in the gastrointestinal tract for chronic physiological monitoring or controlled drug release in the gastrointestinal tract. A self-deploying system includes a substrate, sensor and torsion springs and an endoscopic system includes a tubular device that includes detachable components at the distal portion thereof.

### CROSS-REFERENCE TO RELATED APPLICATIONS

The present application claims priority to U.S. Provisional Application No. 63/169,419 filed on Apr. 1, 2021 and U.S. Provisional Application No. 63/169,439 filed on Apr. 1, 2021, the subject matter of both applications incorporated by reference herein in their entirety.

### TECHNICAL FIELD

The present disclosure relates to endoscopic and self-deployable gastrointestinal suture retention devices and systems.

### BACKGROUND

One of the challenges in the field of chronic physiological monitoring and controlled drug release in the gastrointestinal (GI) tract is achieving prolonged residency in the GI tract. Ingestible electronic devices have the potential to revolutionize the fields of physiological monitoring and controlled drug delivery. However, a primary challenge in the design of ingestible devices for multi-day or multi-week applications is achieving sufficient residency in the GI tract to be therapeutically effective.

### SUMMARY

GI suture retention devices and systems are provided herein. In an aspect, a GI suture retention device can comprise a substrate and at least one torsion spring disposed on the substrate that is configured to transition from a loaded stated to an un-loaded state. A retaining pin can be disposed on the substrate adjacent to the torsion spring that is configured to maintain the torsion spring in a loaded state. A curved needle can be coupled to the torsion spring and can have a first end, an opposing second end comprising a needle tip, and an outer dissolvable needle shaft defining a lumen extending between the first end and the distal tip. The needle shaft can be configured to extend upward from the torsion spring when the torsion spring is in a loaded state and extend downward from the torsion spring when the torsion spring is in an unloaded state. A suture can extend within the lumen of the needle shaft. A needle tip receiving port can be disposed on the substrate and can be sized and configured to accept the needle tip of the curved needle. A sensor can be disposed on the substrate at a location such that the sensor interfaces with an intestinal wall when the device is deployed in the intestine. The sensor can be configured to sense a physiological parameter in the gastrointestinal tract. The device can include a plurality of sutures and accompanying curved needles and torsion springs.

In an aspect, a suture retention system is provided that comprises a proximal handle having a suction source connection port configured to be in fluid communication with a suction source. An outer tube can extend from the proximal handle and can define a suction lumen in fluid communication with the suction source and can comprise a first portion detachably coupled to a distal second portion. The distal second portion can comprise a tissue suction port in fluid communication with the suction lumen, a pierceable barrier, and a wall distal to the pierceable barrier. A push shaft can be configured to be slidably received by the outer tube. A retention shaft can comprise a frangible section connecting the first portion to the second portion of the outer tube. The system can further include a retention device comprising a proximal anchor, a distal anchor, and a suture connected to the proximal anchor at one end and the distal anchor at another end.

### BRIEF DESCRIPTION OF THE DRAWINGS

FIG. 1 is a side view of an exemplary ingestible housing of a self-deployable GI suture retention device according to an aspect of the present disclosure.

FIG. 2 is side schematic view of exemplary internal components of a self-deployable GI suture retention device according to an aspect of the present disclosure.

FIGS. 3A-3C illustrate different configurations of the internal components of the self-deployable GI suture retention device of FIG. 2 during different stages of deployment.

FIG. 4 is a perspective view of exemplary internal components of a self-deployable GI suture retention device according to an aspect of the present disclosure.

FIG. 5 is side schematic view of self-deployable GI suture retention system depicting exemplary internal components of the self-deployable GI suture retention device and an external component according to an aspect of the present disclosure.

FIG. 6 is a top view of an exemplary endoscopic GI suture retention system according to an aspect of the present disclosure.

FIG. 7 is a top view of the distal portion of an endoscopic GI suture retention system according to an aspect of the present disclosure illustrating a first portion of an outer tube coupled to a second portion of the outer tube with a tissue anchor in a loaded configuration according to aspect of the present disclosure.

FIG. 8 is a top view of the distal portion of the system depicted in FIG. 7 when a push shaft has been deployed, urging a distal anchor of the tissue anchor through tissue (not shown) and through a pierceable barrier of the second portion of the outer tube.

FIG. 9 is a top view of the distal portion of the system of FIG. 8 when the push shaft has been retracted and the distal anchor is positioned between a wall of the second portion of the outer wall and the pierceable barrier of the second portion of the outer tube.

FIG. 10 is top view of the distal portion of the system of FIG. 9 when a retention shaft has been pulled proximally, disconnecting the first portion from the second portion of the outer tube.

FIG. 11 is a top view of the second portion of the outer tube of FIG. 10 and the attached tissue anchor after the second portion has decoupled from the first portion of the outer tube.

FIG. 12 is a top view of an exemplary tissue anchor of an exemplary endoscopic GI suture retention system according to an aspect of the present disclosure.

FIG. 13 is a top view of a second portion of an outer tube and a tissue anchor of an exemplary GI suture retention system with a control module attached thereto and an external source in communication with the control module according to an aspect of the present disclosure.

FIG. 14 is a schematic side view of components of the distal portion of an exemplary endoscopic GI suture retention system according to an aspect of the present disclosure.

FIG. 15 a schematic side view of components of the distal portion of an exemplary endoscopic GI suture retention system according to an aspect of the present disclosure after the system has been endoscopically placed in the GI tract.

FIG. 16 is a schematic side view of the components of the distal portion of the system of FIG. 15 when a suction source has been activated and suction is applied through a suction lumen drawing tissue into a suction port.

FIG. 17 is a schematic side view of the components of the distal portion of the system of FIG. 16 after a push shaft has been actuated urging a distal tissue anchor through tissue and through a pierceable barrier.

FIG. 18 is a schematic view of a second portion and tissue anchor of the system of FIG. 17 after the suction source has been deactivated, the push shaft and a retention shaft have been pulled distally releasing the second portion of the outer tube from the first portion.

### DETAILED DESCRIPTION

As used herein with respect to a described element, the terms “a,” “an,” and “the” include at least one or more of the described elements including combinations thereof unless otherwise indicated. Further, the terms “or” and “and” refer to “and/or” and combinations thereof unless otherwise indicated. By “substantially” is meant that the shape or configuration of the described element need not have the mathematically exact described shape or configuration of the described element but can have a shape or configuration that is recognizable by one skilled in the art as generally or approximately having the described shape or configuration of the described element. A “patient” as described herein includes a mammal, such as a human being. The term “top,” “bottom,” “upper,” “lower,” “above,” and “below” refer to the position or orientation of the components as depicted in the drawings. The terms “first,” “second,” etc. are used to distinguish one element from another and not used in a quantitative sense unless indicated otherwise. The term “proximal” refers to a direction/position/orientation towards the patient's mouth and the term “distal” refers to the direction/position/orientation towards the patient's intestines and downstream GI tract. In addition, when an element is referred to as being “on,” “attached” to, “connected” to, “coupled” with, “contacting,” in “communication” with, “extending” from etc., another element, it can be directly on, attached to, connected to, coupled with, contacting, in communication with, or extending from the other element or intervening elements may also be present. In contrast, when an element is referred to as being, for example, “directly on,” “directly attached” to, “directly connected” to, “directly coupled” with, “directly contacting,” in “direct communication” with, or “directly extending” from another element, there are no intervening elements present. An element that is disposed “adjacent” to another element may have portions that overlap or underlie the adjacent element. An “ingestible housing” as used herein is a housing that is not just capable of being ingested but rather is understood by one skilled in the art as be suitable for swallowing and entering into the gastrointestinal tract for therapeutic purposes.

In an aspect, a self-actuating GI suture retention device is provided that is configured to attach to the lining of the GI tracked for an extended period of time (e.g multiple days).

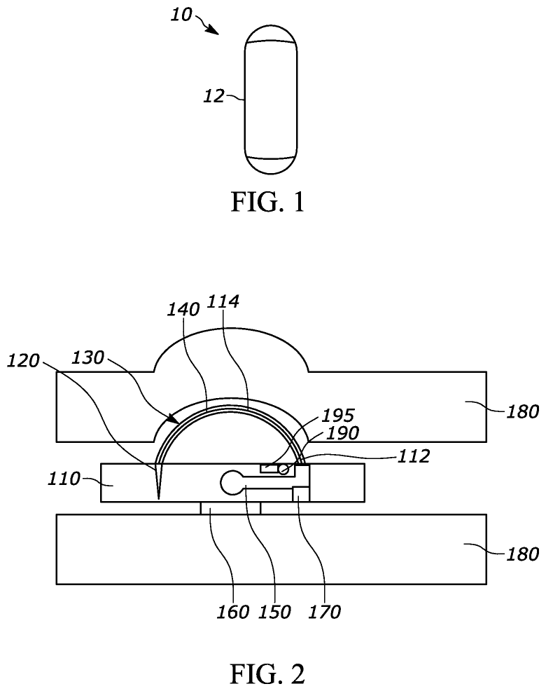

Referring to FIG. 1, device 10 can comprise an ingestible housing 12 that can be in the form of a capsule, tablet, or other suitable configuration having a shape and size suitable for ingestion. The ingestible housing can have an enteric coating so that the device remains intact until it has reached the small intestine. Once reaching the small intestine, the enteric coating can dissolve and the internal components of the device can unfold or unfurl in the lumen of the small intestine, for example. The device can also be configured to reside in other parts of the GI tract including the stomach and large intestines. FIG. 2, illustrates exemplary internal components that can be contained within the ingestible housing of the device. The device can include substrate 110 and torsion spring 150 disposed on substrate 110 and configured to transition from a loaded stated to an un-loaded state. Retaining pin 190 can be disposed on substrate 110 adjacent to torsion spring 150 (such as above the torsion spring) and can be configured to maintain torsion spring 150 in a loaded state. Curved needle 130 can be coupled to torsion spring 150 and can comprise one end 112, an opposite end comprising a needle tip 120, and outer dissolvable needle shaft 114 defining a lumen extending between end 112 and needle tip 120. Needle shaft 114 can be configured to extend upward from torsion spring 150 when torsion spring 150 is in a loaded state and extend downward from torsion spring 150 when torsion spring 150 is in an unloaded state. Needle shaft 114 is illustrated in the figures as having a semi-circular shape but it can have other suitable shapes so long as it can transition to a position within GI tissue wall 180. Flexible suture 140 can extend within the lumen of needle shaft 114. Needle tip receiving port 170 can be disposed on substrate 110 and can be sized and configured to accept needle tip 120 of curved needle 130. Sensor 160 can be disposed on substrate 110 at a location such that the sensor interfaces with intestinal wall 180 when the device is deployed in the intestine or other part of the GI tract. Sensor can be configured to sense a physiological parameter in the GI tract and can include, for example, an photoplethysmography (PPG) sensor, an accelerometer, a gyroscope, an electrocardiogram (ECG) sensor, a temperature sensor, or combinations thereof. The sensor can be configured to detect a physiological parameter indicative of a medical condition. A controller can be operatively coupled to the sensor and can be programmed to receive the physiological parameter and prepare the physiological parameter for transmission from the subject, analysis of the physiological parameter, or both.

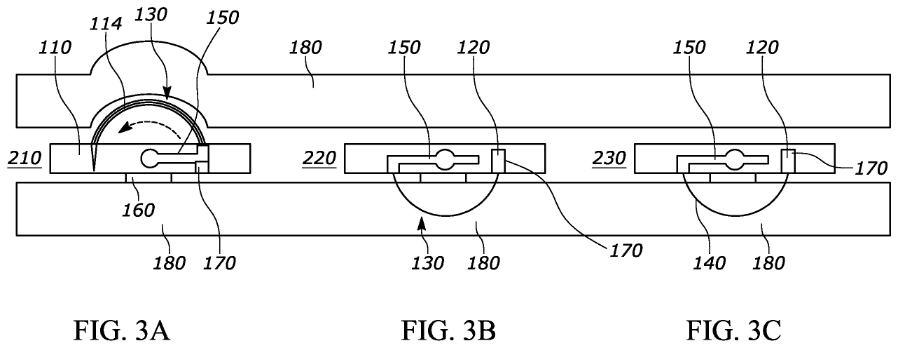

FIG. 3A-3C schematically illustrates the configuration of the device during different stages of deployment. At stage 210, torsion spring 150 can be actuated when sensor 160 detects contact with intestinal wall 180 thereby triggering displacement of the retaining pin and resultant release of torsion spring 150 from a loaded state (depicted in FIG. 3A) to an un-loaded state (depicted in FIGS. 3B and 3C). The retaining pin can be displaced in different ways. For example, the substrate can include an electromagnet 195 that can supply power sufficient to displace the retaining pin away from the torsion spring, releasing the torsion spring. At stage 220 illustrated in FIG. 3B, when the potential energy of torsion spring 150 is released, curved needle 130 rotates relative to substrate 110, pushing curved needle 130 through a segment of intestinal tissue 180 in its path until needle tip 120 re-emerges out of the tissue and is received by receiving port 170. The needle shaft 114 can comprise a dissolvable material that breaks down over the time course of, for example, 10-100 minutes. When curved needle 130 dissolves, flexible suture 140 can remain, mechanically coupling substrate 110 to tissue 180 at stage 230 depicted in FIG. 3C. When deploying, the mechanism mimics an attachment of the device to tissue similar to what would be achieved with standard surgical suturing technique, with a sharp needle tip piercing into tissue, drawing a flexible suture through the tissue and emerging out of the tissue. With two points of attachment onto the substrate, with the suture traveling through a segment of intestinal tissue, the device is held in place anchored to the intestinal tissue by the suture. As such, the device does not require an endoscopic, percutaneous or open surgical procedure to anchor the device to GI tissue. The flexible suture may also be dissolvable, with the absorption time of the suture chosen to control the duration of intestinal residency. When the suture weakens as it is absorbed, the interface between the suture and the device disconnects, thereby releasing the device and allowing it to pass naturally through the GI tract.

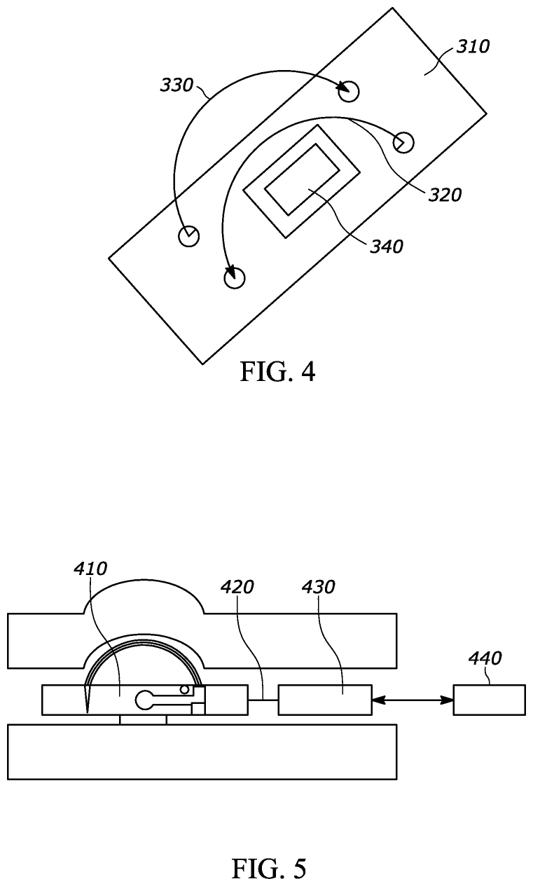

In another aspect, multiple spring-loaded curved needles can be integrated into a single retention device. Referring to FIG. 4, substrate 310 can comprise a plurality of curved needled that are deployed in opposing directions using the components described above. FIG. 4 illustrates two curved needles 320, 330 that are deployed in opposing directions (curved needle 330 can rotate clockwise while curved needle 320 can rotate counterclockwise, or vice versa). Although FIG. 4 only illustrates two needles, any number of suitable needles can be included. The potential advantage of multiple needles is to improve tissue contact between an optical sensor, such as a PPG sensor 340, and the intestinal tissue. Another advantage is the robustness of the connection given that there are multiple points of connection to the tissue.

Referring to FIG. 5, substrate 410 may be mechanically coupled via a linker, cable or other device 420 to module 430 comprising other components in the ingestible device, including but not limited to a controller, physiological sensors (e.g. PPG, accelerometer, acoustic, temperature sensors), batteries, radio for wireless data transmission, and a drug delivery dispenser. This device can be used for a variety of clinical applications requiring prolonged residency in the GI tract, including responsive drug delivery use cases in which physiological sensors monitor physiological biomarkers and responsively deliver a drug to treat a medical condition. One such application is detection and reversal of opioid overdose in which the physiological sensors detect respiratory depression caused by opioid overdose and release an opioid antagonist to reverse or mitigate the overdose. Devices disclosed herein provide components to retain the device in the intestine to provide continuous monitoring of such a life-threatening clinical event.

In particular, in certain aspects, substrate 410 can be in communication with control module 430, that can include, for example, a microprocessor or controller to process sensor data, a battery to power the system, a radio/antenna to wirelessly communicate data with a receiver/external device 440 outside the body, or combinations thereof. Although FIG. 5 only depicts one module, these different components could be part of separate module(s) in communication with other module(s). For example, the rest of the electronics could be on another PCB or multiple PCBs in a module tethered to the substrate. The controller can be operatively coupled to the substrate/sensor and can be programmed to receive the physiological parameter measured by the sensor that is indicative of a medical condition and prepare the physiological parameter for transmission from the subject, analysis of the physiological parameter, or both.

The controller can be operably coupled to a drug dispenser to actuate release of drug to mitigate the medical condition. For example, the controller can be programmed to actuate release of a medication from the drug dispenser into the patient in response to a determination that the physiological parameter falls outside a threshold value or range for the at least one physiological parameter. The controller can also be programmed to preemptively actuate release of the medication from the drug dispenser into the patient in response to the determination such that the medication is delivered prior to the patient experiencing the medical condition. The sensor can be configured to detect an individual physiological parameter, a combination of physiological parameters, trends in an individual physiological parameter, or trends in a plurality of physiological parameters. The controller can be programmed to actuate release of the medication from the drug dispenser into the patient in response to a determination that the individual physiological parameter, the combination of physiological parameters, trends in the individual physiological parameter, or trends in the plurality of physiological parameters meet a pre-defined condition. The controller can also be programmed to preemptively actuate release of the medication from the drug dispenser into the patient in response to the determination such that the medication is delivered prior to the patient experiencing the medical condition.

For example, the controller can be programmed to send a control signal to a drug delivery dispenser as part of the device or system in response to a measurement of a physiological signal by the sensor that deviate beyond clinically established unsafe levels. One example of this application is a pulse oximeter sensor integrated into the device to measure blood oxygen saturation, heart rate, respiratory rate, or combinations thereof from within the GI tract. In the case of an opioid overdose, the device can detect changes in physiological values (e.g. a decrease in blood oxygen saturation and/or a decrease in respiratory rate) and command a drug delivery dispenser to release an opioid antagonist (e.g. nalmefene or naloxone) to mitigate the effects of the overdose to prevent serious injury or death. In particular, upon the sensor detecting a physiological indication of an opioid overdose, the drug dispenser can release a rescue medication as well as send out alerts to the patient and/or a caregiver. Non-limiting examples of suitable drug dispensers include MEMS drug delivery, valve systems, osmotic plug pistons, electrolytical pumps, or combinations thereof. In certain aspects, the drug dispenser can be a non-refillable reservoir such that it does not include any ports or similar structures that allow the drug dispenser to be re-filled from a location external to the patient's body, such as a syringe containing an opioid antidote that is injected into the drug dispenser from outside the patient's body. The device can be used for other indications and medications and opioid overdose and an opioid antidote is provided as one example.

The device can include a radio that is configured to communicate with external device 440, wherein the controller is configured and programmed to control the operation of the radio to send an alert to external device 440 in response to determining that the subject is experiencing a medical condition. In certain aspects, the controller can be configured and programmed to control the operation of the radio to preemptively send an alert to the external device in response to receiving the physiological parameter such that alert is sent prior to the subject experiencing the medical condition.

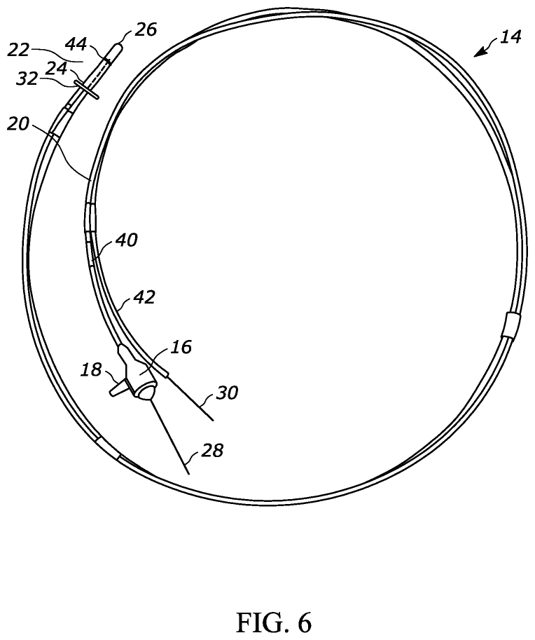

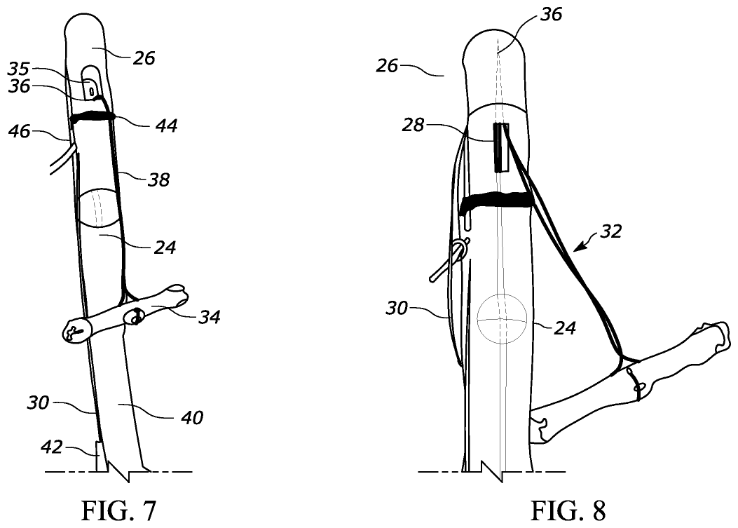

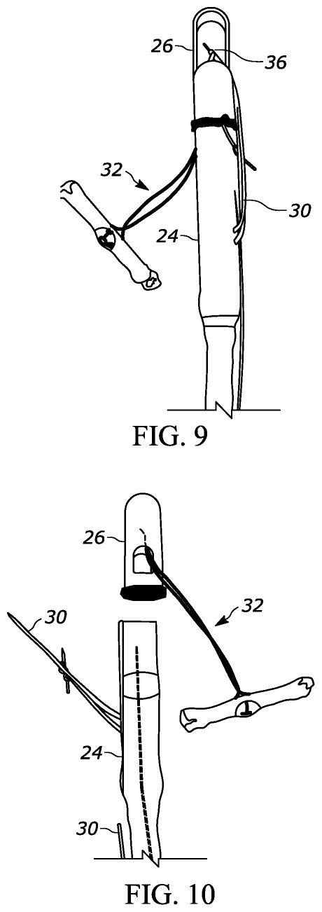

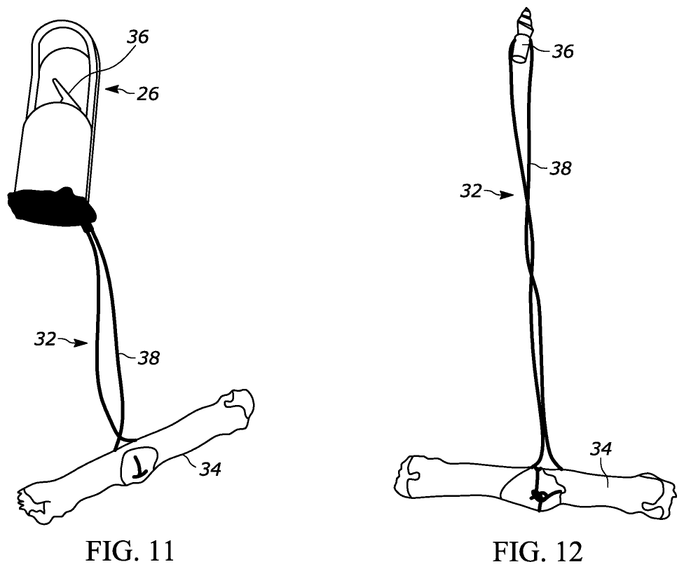

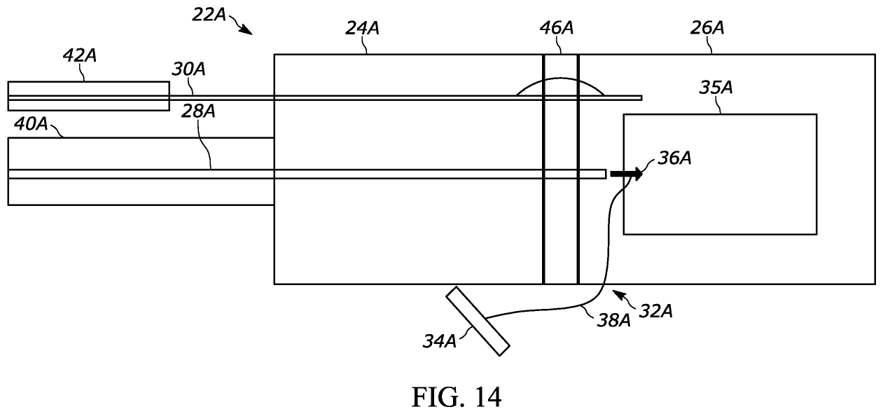

In another aspect, an endoscopic gastrointestinal suture retention system is provided that facilitates multi-day gastrointestinal residency of a device by using a combination of suction and mechanical push force to place a suture through the lining of the GI tract. Such a system can be used without direct visualization. Referring to FIGS. 6-14, in an aspect a system 14 can comprise proximal handle 16 having a suction source connection port 18 configured to be in fluid communication with a suction source. Outer tube 20 can extend from proximal handle 16 and can define a suction lumen in fluid communication with the suction source. Distal portion 22 of outer tube 20 can have first portion 24 detachably coupled to second portion 26, which is distal to first portion 24 as shown in FIG. 6. Second portion 24 can comprise a tissue suction port 35 in fluid communication with the suction lumen, a flexible pierceable barrier, and a wall distal to the pierceable barrier. The pierceable barrier can be fabricated from any material that can be pierced by the distal anchor such as silicone, for example. Gasket 44 can be disposed between first portion 24 and second portion 26 of outer tube 20 to maintain a vacuum in second portion 26 when suction is applied. System 14 can further comprise push shaft 28 configured to be slidably receivable by outer tube 20. Retention shaft 30 with frangible section 46 can connect first portion 24 to second portion 26 of outer tube 20. The frangible section can be an intentionally weaker section than the rest of the retention shaft such that when the retention shaft is pulled proximally with sufficient force, the frangible section fails and breaks the second portion free from the first portion. The frangible section can be a monofilament, for example, while the rest of the retention shaft can be a metallic wire. In certain aspects, and as illustrated in FIG. 1, the push shaft can be slidably receivable by push shaft tube 40 that extends through outer tube 20. Similarly, the retention shaft can be slidably receivable by retention shaft tube 42. The retention shaft tube can extend outside the outer tube as illustrated in FIG. 1 or can extend through the outer tube, as illustrated in FIG. 14. System 14 can further include retention device 32. FIG. 12 illustrate the components of retention device 32, which can be proximal anchor 34, distal anchor 36, and suture 38 connected to proximal anchor 34 at one end and distal anchor 36 at another end. The distal anchor can have an arrowhead configuration as shown in the figures or any other suitable configuration that allows the distal anchor to pierce the pierceable barrier of the second portion of the outer tube. The suture can be attached to approximately the middle portion of the distal anchor. The full system can be delivered through an outertube that provides a path from the mouth to the GI tract, including the stomach, small intestine, or large intestine. An endoscope can be used to place the overtube into the GI tract.

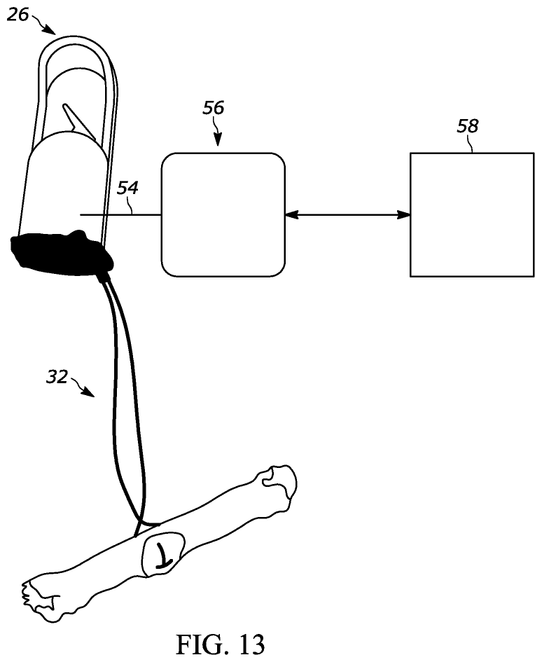

Referring to FIG. 13, second portion 26 can be mechanically coupled via a linker, cable or other device 54 to another module 56 comprising other components, including but not limited to a controller, physiological sensors (e.g. PPG, accelerometer, acoustic, temperature sensors), batteries, radio for wireless data transmission, and a drug delivery dispenser. This device can be used for a variety of clinical applications requiring prolonged residency in the GI tract, including responsive drug delivery use cases in which physiological sensors monitor physiological biomarkers and responsively deliver a drug to treat a medical condition. One such application is detection and reversal of opioid overdose in which the physiological sensors detect respiratory depression caused by opioid overdose and release an opioid antagonist to reverse or mitigate the overdose. Devices disclosed herein provide components to retain the device in the intestine to provide continuous monitoring of such a life-threatening clinical event.

Referring to FIG. 13, in certain aspects, a sensor of the device can be in communication with control module 56, that can include, for example, a microprocessor or controller to process sensor data, a battery to power the system, a radio/antenna to wirelessly communicate data with a receiver/external device 58 outside the body, or combinations thereof. Although FIG. 13, only depicts one module, these different components could be part of separate module(s) in communication with other module(s). For example, the rest of the electronics could be on another PCB or multiple PCBs in a module tethered to the second portion of the outer tube. The controller can be operatively coupled to a sensor and can be programmed to receive the physiological parameter measured by the sensor that is indicative of a medical condition and prepare the physiological parameter for transmission from the subject, analysis of the physiological parameter, or both.

The controller can be operably coupled to a drug dispenser to actuate release of drug to mitigate the medical condition. For example, the controller can be programmed to actuate release of a medication from the drug dispenser into the patient in response to a determination that the physiological parameter falls outside a threshold value or range for the at least one physiological parameter. The controller can also be programmed to preemptively actuate release of the medication from the drug dispenser into the patient in response to the determination such that the medication is delivered prior to the patient experiencing the medical condition. The sensor can be configured to detect an individual physiological parameter, a combination of physiological parameters, trends in an individual physiological parameter, or trends in a plurality of physiological parameters. The controller can be programmed to actuate release of the medication from the drug dispenser into the patient in response to a determination that the individual physiological parameter, the combination of physiological parameters, trends in the individual physiological parameter, or trends in the plurality of physiological parameters meet a pre-defined condition. The controller can also be programmed to preemptively actuate release of the medication from the drug dispenser into the patient in response to the determination such that the medication is delivered prior to the patient experiencing the medical condition.

For example, the controller can be programmed to send a control signal to a drug delivery dispenser as part of the device or system in response to a measurement of a physiological signal by the sensor that deviate beyond clinically established unsafe levels. One example of this application is a pulse oximeter sensor integrated into the device to measure blood oxygen saturation, heart rate, respiratory rate, or combinations thereof from within the GI tract. In the case of an opioid overdose, the device can detect changes in physiological values (e.g. a decrease in blood oxygen saturation and/or a decrease in respiratory rate) and command a drug delivery dispenser to release an opioid antagonist (e.g. nalmefene or naloxone) to mitigate the effects of the overdose to prevent serious injury or death. In particular, upon the sensor detecting a physiological indication of an opioid overdose, the drug dispenser can release a rescue medication as well as send out alerts to the patient and/or a caregiver. Non-limiting examples of suitable drug dispensers include MEMS drug delivery, valve systems, osmotic plug pistons, electrolytical pumps, or combinations thereof. In certain aspects, the drug dispenser can be a non-refillable reservoir such that it does not include any ports or similar structures that allow the drug dispenser to be re-filled from a location external to the patient's body, such as a syringe containing an opioid antidote that is injected into the drug dispenser from outside the patient's body. The device can be used for other indications and medications and opioid overdose and an opioid antidote is provided as one example.

The device can include a radio that is configured to communicate with external device 58, wherein the controller is configured and programmed to control the operation of the radio to send an alert to external device 58 in response to determining that the subject is experiencing a medical condition. In certain aspects, the controller can be configured and programmed to control the operation of the radio to preemptively send an alert to the external device in response to receiving the physiological parameter such that alert is sent prior to the subject experiencing the medical condition.

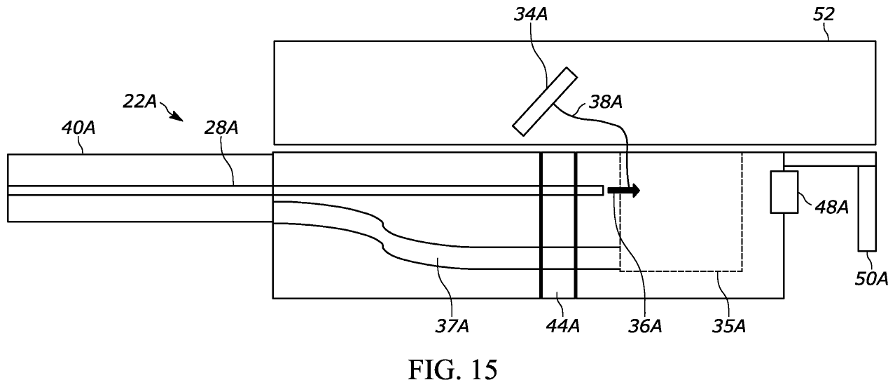

FIGS. 14-17 are schematic side views of internal components of distal portion 22 of a GI suture retention system according to an aspect of the present disclosure. FIGS. 14-15 illustrate first portion 24A detachably coupled to second portion 26A. Second portion 26A includes issue suction port 35A that is in fluid communication with suction lumen 37A. In this aspect, the suction lumen is defined by push shaft tube 40A but could be a separate lumen. Push shaft 28A extends through push shaft tube 40A and is positioned in the outer tube such that it can contact distal anchor 36A when tissue anchor 32A is loaded into second portion 26A of the outer tube to urge distal anchor 36A distally through suction port 35A and through pierceable barrier 48A. In this aspect, retention shaft 30A is slidable received by retention shaft tube 42A, which is within the outer tube. As stated above, all or a part of the retention shaft can extend outside of the outer tube. Frangible section 46A of retention shaft is depicted in FIG. 14 as being a monofilament, but could be fabricated from another material or comprise another component so long as the frangible section breaks when the retention shaft is pulled proximally with sufficient force to release second portion 26A from first section 24A.

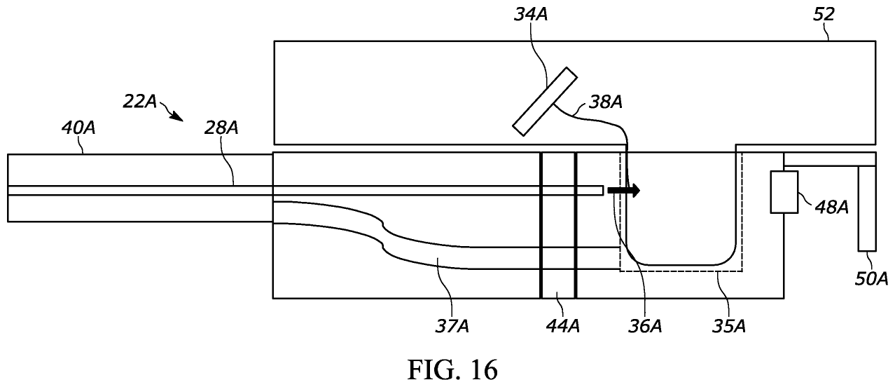

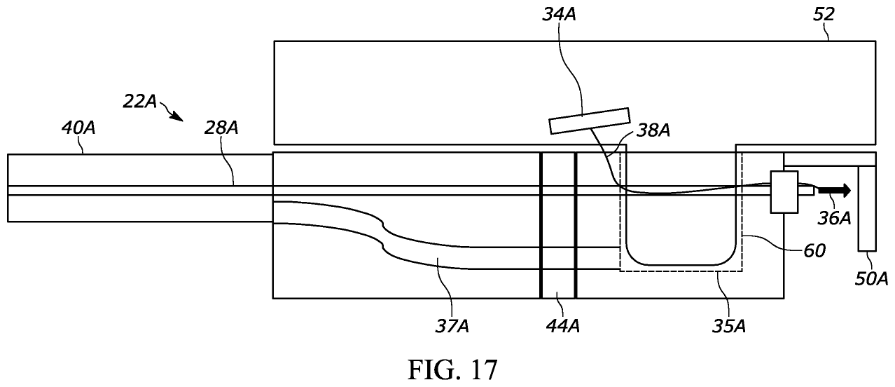

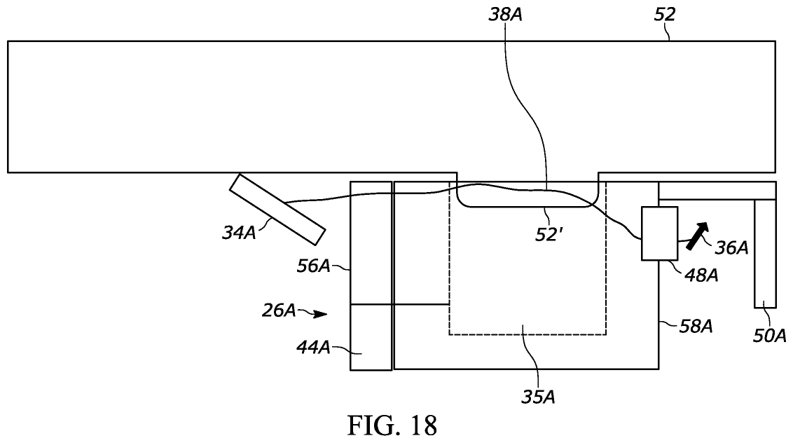

FIG. 15 illustrated distal portion 22A of the outer tube when the system has been endoscopically placed in the GI tract (FIGS. 15-17 do not depict the retention shaft or retention shaft tube illustrated in FIG. 14 for purposes of clarity). FIG. 16 illustrates the position and configuration of components of distal portion 22A when the suction source at the proximal end of the system has been activated delivering suction through suction lumen 37A into suction port 35 and thereby drawing tissue 52A into tissue suction port 35A. FIG. 17 illustrates the position and configuration of components of distal portion 22A when push shaft 28A has been pushed distally to engage with and urge distal anchor 36A through tissue suction port 35A and through pierceable barrier 48A and towards wall 50A, which can prevent distal anchor 36A from advancing any further distally. FIG. 18 illustrates the position and configuration of components of distal portion 22A when the suction source has been deactivated, the push shaft and retention shaft have been drawn back proximally. At this stage, the retention shaft breaks at its frangible section, decoupling the second portion 24A from the first portion of the outer tube. As seen in FIG. 18, suture 38A extends through segment of tissue 52′. Proximal anchor 34A prevents suture 38A from sliding out of proximal end 56A of second portion 26A and distal anchor 36A is confined to a region between barrier 48A and wall 50A thus preventing suture 38A from sliding out of distal end 58A of second portion 26A.

The proximal portion of the distal anchor can be a hollow tube and the distal end can be a closed solid tip with a sharp edge. The hollow tube of the distal anchor can be configured to receive the distal end of the push shaft. When the push shaft slidably enters the hollow tube of the distal anchor it can urge the distal anchor through the pierceable barrier. When the distal anchor hits wall 50A of the second portion of the outer tube, the distal anchor can rotate and assume a “T-shape.” The opening 60 between suction port 35A and beyond it distally can be larger than the width of the distal anchor, but not the length of the distal anchor such that when the distal anchor rotates, it does not get drawn back into the suction port. The pierceable barrier that plugs the opening of the suction port can only open enough for the distal anchor to pass through it, and because it is a flexible barrier (such as silicone, for example), it can close back slightly. Thus, even if the distal anchor remained perfectly aligned with the longitudinal plane of insertion and travel, it may not be drawn back proximally through the flexible pierceable barrier. However, the distal anchor will generally rotate because the suture is preferably attached at substantially the midpoint of the distal anchor and the force acting on distal anchor is generally not always perfectly along the axis of motion of the push shaft. Since the distal anchor rotates in such embodiments, even if enough force was applied that could draw the distal anchor through the pierceable barrier, the distal anchor would not pass back through the opening of the suction port because the length of the distal anchor cannot fit through this opening.

The suction connect port can be fully deattached from the suction source and tissue anchor 38A couples second portion 24A to the tissue. The suture can be dissolvable to predetermine the duration of the residency of the device. By choosing a suture material with a known absorption time, as the suture is absorbed, it will eventually disconnect from the tissue anchor releasing the device.

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. A gastrointestinal suture retention system comprising:
a proximal handle having a suction source connection port configured to be in fluid communication with a suction source;
an outer tube extending from the proximal handle and defining a suction lumen in fluid communication with the suction source and comprising a first portion detachably coupled to a distal second portion, the distal second portion comprising a tissue suction port in fluid communication with the suction lumen, a flexible pierceable barrier, and a wall distal to the flexible pierceable barrier;
a push shaft configured to be slidably received by the outer tube;
a retention shaft comprising a frangible section connecting the first portion to the second portion of the outer tube;
a retention device comprising a proximal anchor, a distal anchor, and a suture connected to the proximal anchor at one end and the distal anchor at another end.

2. The suture retention system of claim 1, wherein the distal anchor has an arrowhead shape.

3. The suture retention system of claim 1, further comprising a push shaft tube extending through the outer tube that is configured to slidably receive the push shaft.

4. The suture retention system of claim 1, further comprising a retention shaft tube extending outside the outer tube and that is configured to slidably receive the retention shaft.

5. The suture retention system of claim 1, further comprising a retention shaft tube extending through the outer tube and that is configured to slidably receive the retention shaft.

6. The suture retention system of claim 1, wherein the suture is attached to approximately the middle portion of the distal anchor.

7. The suture retention device of claim 1, wherein the proximal portion of the distal anchor is a hollow tube and the distal portion is a closed, solid tip with an edge sufficiently sharp to pierce the flexible pierceable barrier.

8. The suture retention system of claim 1, wherein a gasket is disposed between the first portion and the second portion of the outer tube.

9. The suture retention system of claim 1, wherein the second portion of the outer tube comprises a sensor configured to sense a physiological parameter in the gastrointestinal tract.

10. The device of claim 9, further comprising a controller operatively coupled to the sensor, the controller programmed to receive the physiological parameter and prepare the physiological parameter for transmission from the subject, analysis of the physiological parameter, or both.

11. The device of claim 10, wherein the device comprises a drug dispenser operably coupled to the controller, the controller programmed to actuate release of a medication from the drug dispenser into the patient in response to a determination that the physiological parameter falls outside a threshold value or range for the at least one physiological parameter or in response to a determination that the physiological parameter meets a pre-defined condition.

12. The device of claim 1, wherein the suture is fabricated from a dissolvable material.

13. The device of claim 1, wherein the frangible section of the retention shaft is a monofilament.

14. A gastrointestinal suture retention system comprising:
the device of claim 1;
a radio configured to communicate with an external device; and
a controller programmed to control the operation of the radio to send an alert to the external device in response to determining that the subject is experiencing a medical condition.

15. A gastrointestinal suture retention system comprising:
a substrate;
a torsion spring disposed on the substrate and configured to transition from a loaded stated to an un-loaded state;
a retaining pin disposed on the substrate adjacent to the torsion spring configured to maintain the torsion spring in a loaded state;
a curved needle coupled to the torsion spring and comprising a first end, an opposing second end comprising a needle tip, and an outer dissolvable needle shaft defining a lumen extending between the first end and the distal tip, the needle shaft configured to extend upward from the torsion spring when the torsion spring is in a loaded state and extend downward from the torsion spring when the torsion spring is in an unloaded state;
a suture extending within the lumen of the needle shaft;
a needle tip receiving port disposed on the substrate and sized and configured to accept the needle tip of the curved needle; and
a sensor disposed on the substrate at a location such that the sensor interfaces with an intestinal wall when the device is deployed in the intestine, the sensor configured to sense a physiological parameter in the gastrointestinal tract.
